(19) United States
(12) Patent Application Publication
(10) Pub. No.: US 2009/0023600 A1
(43) Pub. Date: Jan. 22, 2009

(54) Device and Process for Measuring Cell Properties

(75) Inventors: Richard Wagner, Bad Iburg (DE); Karsten Gall, Lunestedt (DE); Andreas Wirth, Telgte (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Universitat Osnabruck, Osnabruck (DE)

(21) Appl. No.: 11/718,616

(22) PCT Filed: Nov. 4, 2005
(86) PCT No.: PCT/EP05/55752

§ 371 Date: Jun. 16, 2008

(30) Foreign Application Priority Data
Nov. 5, 2004 (EP) 04026252.9
Dec. 20, 2004 (EP) 04030135.0

(51) Int. Cl. C40B 40/02; C12M 1/00; C12Q 1/02

(52) U.S. Cl. 506/14; 435/287.1; 435/288.7; 435/29

(57) ABSTRACT

The invention relates to a measuring device for measuring at least one property of cells or vesicles, characterized in that is divided into two chambers by a hydrophobic partition having a horizontal opening, these chambers containing electrolyte solution and said opening being closed by a lipid double layer. The invention is characterized in that at least one cell or vesicle contacts the lipid double layer. The invention also relates to methods involving the use of the measuring device and to arrays, measuring chambers and microtitration plates all suited therefor.

The invention relates to processes for establishing properties of cells and vesicles, wherein at least one cell or vesicle contacts a lipid bilayer. The invention also relates to measuring devices for use in said processes.

For establishing electrophysiological properties of cells, it is common to use the patch-clamp technology. Thus, a glass pipette (about 1 μm in diameter) is filled with an electrolyte solution and carefully placed on the surface of a cell. Then, after having perforated the membrane patches, currents or the potential over the whole cell surface can be measured in the patch pipette (whole-cell recording). However, the actual advantage of the patch clamp technology is the ability to measure currents through individual channels present in the membrane patch directly below the pipette tip. In a method for the partial automation of this technology, a cell is sucked onto a smaller opening between two chambers being on top of one another, wherein the opening must be adapted to the cell size. When the cell has thus been stabilized and the opening sealed thereby, electric measurements can be effected as long as the cell-opening connection is substantially sealed for ions (seal). The sealing resistance between the cell and the edge of the opening is on the order of 10⁹ Ω; this is referred to as a gigaseal. This temporally stable gigaseal, which is absolutely necessary for a high resolution (pA−nA), is the main problem in the automation of patch clamp measurements. With the current method of sucking a cell onto a smaller opening, sealing resistances within a range of from 10⁷ to 10⁸ Ω are achieved, and in addition, they are not stable in time. A reproducibly high sealing resistance can be achieved only in an individual experiment, and only with a smaller number of membrane types, which strongly limits the applicability of the patch clamp technology. For the modern research into active substances, this means that, in spite of various attempts of automation, as yet non-standardized methods, predominantly based on fluorescence, must still be recurred to. For the approval of medicaments, in which pharmacological safety enjoys the highest priority, for medicaments that must be tested almost exclusively with electrophysiological measuring techniques, sequential individual measurements are a considerable obstacle. The screening of potentially active substances on a large scale is not possible with this method. Optical measurements cannot be performed with such automated patch clamp systems either.

Another established technology for establishing electrophysiological data includes vertical lipid bilayers spanned between two chambers filled with electrolyte (Borisenko et al., 2003; Hinnah et al., 2002). After fusion of ion channels or transporter proteins into the lipid bilayers, the current mediated by the proteins can be resolved to the single molecule level. The established classical lipid bilayer technology cannot be automated. In contrast to the patch clamp technology, it is not possible thereby to measure the properties of membranes of intact cells. Therefore, this technology is not suitable or approved for the testing of pharmacologically active substances, because only measuring methods performed on whole cells are employed for this purpose.

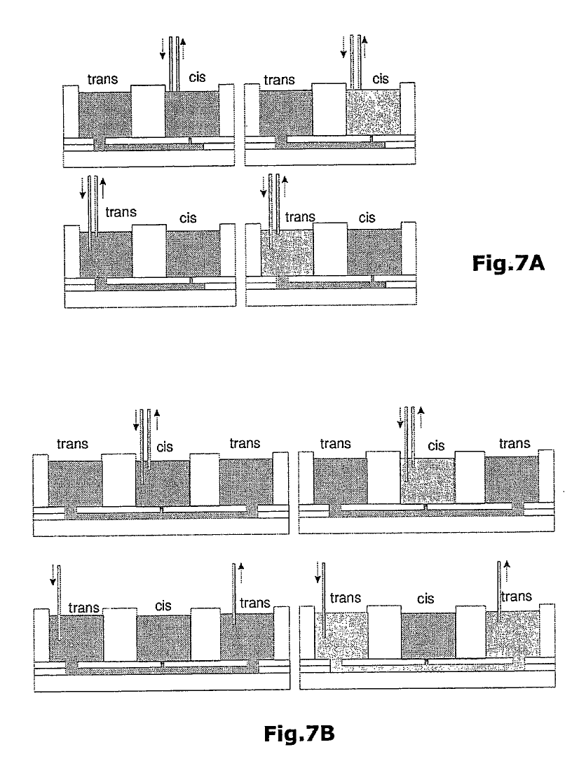

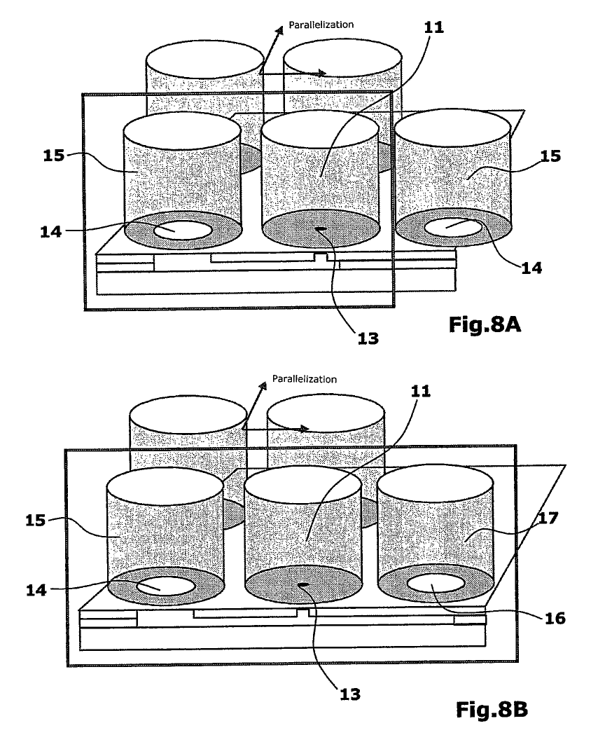

US 2003/0146091 A1 describes a device for performing measurements on cells with one or more samples. In embodiments with several measuring chambers (FIGS. 7 and 8), the cell is bonded to an opening which is positioned in a hydrophilic partition wall made of, for example, silicon nitride or silica. The hydrophilic partition wall is in part coated with a hydrophobic coating in order that the cell can be positioned in such a way that its membrane joins the hydrophobic coating. The preparation of such multilayered device is comparatively tedious.

Said positioning of the cells on the device is error-prone, since the measuring object itself, i.e., the cells, must first seal all openings of the device to achieve a “gigaseal” and thus to be able to perform measurements.

Samsonov et al., 2002, examine the fusion of lipid bilayers containing fusion factors with cells that express the cell fusion proteins on their surface. A measuring chamber having a hydrophobic partition wall containing an opening of 150-200 μm is used. Into the opening, a lipid bilayer is inserted which contains factors essential to the fusion, such as cholesterol and sphingolipids. The establishing of this system is tedious since the special fusion factors must be added and expressed.

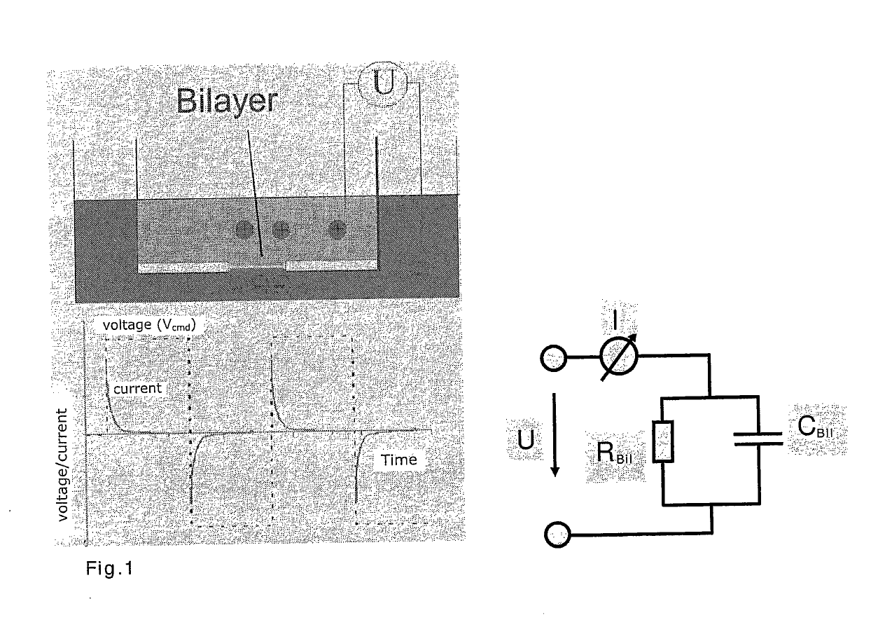

DE 10047390 A1describes devices having a plurality of measuring chambers which are respectively separated by a lipid bilayer. After applying a voltage across the lipid bilayers, changes of the membranes upon the addition of organic compounds can be examined. The device is suitable for high-throughput screening. However, the device has drawbacks: Thus, the disassembling of the device and the application of the bilayers according to FIG. 1b is cumbersome. The lower sample chambers are not readily accessible and only with difficulty can they be filled uniformly and cleaned. The silver electrodes are arranged in such a way that optical measurements are not possible. Since the lower chambers are completely closed after the lipid bilayer has been formed, an osmotic pressure which adversely affects the membranes and thereby distorts the measurements can be generated when samples are added to the chamber above the membrane.

Hanju et al., 1998, describe examinations of vertically arranged lipid bilayers in a single measuring chamber. An examination of cells or vesicles is not performed and, because of the arrangement of the measuring chamber, would be possible only if such cells and vesicles were fixed to the horizontal membrane by particular means.

Bellemare et al., 1996, examine ion channels in lipid bilayers. A Delrin cup, i.e., essentially a smaller vessel with a vertical measuring opening within a larger vessel, is used as the measuring assembly, wherein the opening has a diameter of from 200 to 400 μm.

Sanford et al., 1989, also describe examinations of lipid bilayers in a single measuring chamber on a vertical membrane.

It is the object of the present invention to develop a novel microanalytical or nanoanalytical technology which overcomes the drawbacks of the above described methods. In particular, the object is to provide a process and means for establishing cell properties, such as electrophysiological and optical properties, exactly, reliably and reproducibly in as simple a way as possible. The process is to be apt for automation and for use already in the early primary screening of active substances.

The object of the invention is surprisingly achieved by devices and processes according to any of claims 1 to 27.

The invention relates to a measuring device for establishing at least one property of cells or vesicles, partitioned by a hydrophobic partition wall having a horizontal opening (measuring opening) into two chambers containing an electrolyte solution, wherein said opening is closed by a lipid bilayer. At least one cell or one vesicle is in contact with the lipid bilayer.

A lipid bilayer is spanned across the opening. This lipid bilayer forms spontaneously when a biological, purified, naturally occurring or synthetic lipid in a suitable form is applied to a hydrophobic substrate. Further, the lipid bilayer seals the opening and thus the two chambers from one another with a resistance of typically around 10¹⁰ Ω. The stability of the lipid bilayer over time is inversely proportional to the radius of the opening and is within a range of hours to days already for holes with a diameter of 30 μm (Hinnah et al., 2002).

If a biological cell is contacted with the lipid bilayer, their electrical properties become coupled. Said contacting can be effected, for example, by the lipid bilayer being arranged horizontally. When the cell or vesicle is then added onto or above the lipid bilayer, it comes into contact with the lipid bilayer mainly by gravity. However, the contacting can also be effected or enhanced in another way, for example, by moving the measuring device, the electrolyte solution and/or by making use of attractive interactions between the lipid bilayer and the cell or vesicle. For example, the contacting can also be effected or enhanced by using suitable receptor/ligand systems or surface proteins which promote cell fusion. However, such specific interactions are not required for the system according to the invention. In a preferred embodiment of the invention, the cell/membrane fusion is not artificially enhanced by adding corresponding fusion components or by the previous well-aimed expression of protein components which effect or promote the fusion, such as the E1 and E2 envelope proteins. Rather, solely by adding the vesicles to a lipid bilayer, conditions which in a simple way allow the measurement of the vesicle properties are surprisingly produced.

The molecular mechanisms of coupling are not known in detail, but their result is a coupling of the cell with the lipid membrane in which the cell essentially maintains its morphological structure and represents a common electric resistance together with the bilayer (conductive coupling). In a preferred embodiment, the lipid membrane of the cell or vesicle fuses partially or completely with the lipid bilayer after contacting it. The components of the cell or vesicle membrane, for example, the ion channels, thereby become components of the lipid bilayer barrier which partitions the chambers. The establishing of the cell properties is preferably effected after the fusion is completed and the system has stabilized.

However, it is to be pointed out that the process according to the invention can be applied even when a cell or vesicle contacts a lipid bilayer and both form a common capacitor (capacitive coupling) without a fusion of the membranes occurring. Such an arrangement already allows, for example, the measurement of electrical properties of the cell, which serves as a capacitor when a voltage is applied across the lipid bilayer.

In a preferred embodiment, the horizontal opening (measuring opening) has a diameter of from 0.1 to 100 μm, especially from 0.5 to 50 μm or from 2 to 40 μm. The opening is preferably round or oval in shape. The lipid bilayer which is positioned within the opening is essentially horizontal due to the opening. This means that it may be slightly curved upwards or downwards. Such a curve can be formed, for example, when the solutions above and below the membrane are different.

The hydrophobic partition wall may, but need not be horizontal. For example, it may have a slight inclination towards the measuring opening in order that a cell added to the first cavity (11) gets towards the lipid bilayer by gravity. In preferred embodiments, the inclination of the hydrophobic partition wall with respect to level is less than 45, 20 or 10%.

The hydrophobic partition wall is preferably a polymer film. In particular embodiments of the invention, the partition wall is made of Teflon, PMMA, PDMS, Topas or polycarbonate. The partition wall preferably has a thickness of 1-100 μm. Suitable materials generally include those which are sufficiently non-polar, so that a lipid bilayer can be stably introduced in an opening in the material. In connection with the partition wall, “hydrophobic” means that a stable lipid bilayer can be formed in an opening in the partition wall. The electrolyte solution is preferably physiological saline. Suitable salts include those, in particular, which contain the ions K⁺, Na⁺, Mg²⁺, Ca²⁺, Cl⁻, SO₄²⁻ and PO₄³⁻. The pH value is preferably between 5 and 9 and is adjusted with suitable pH buffering substances.

The lipid bilayer is made, in particular, of synthetic and/or purified biological lipids. The production of lipid bilayers in openings in partition walls for performing electrophysiological measurements is known from the prior art, for example, from Hinnah et al., 2002, which is included herein by reference. Preferably, a lipid preparation of synthetic or purified biological lipids is used for producing the lipid bilayer. In such a lipid preparation, the lipids are no longer in their natural environment, i.e., they are no longer components of the intact cell organelle, cell or other naturally occurring vesicle.

In a preferred embodiment of the invention, a circuit has been applied through the measuring device and through the lipid bilayer, so that measurements with respect to electrical properties of the lipid bilayer and of the cells in contact with the lipid bilayer can be performed.

In another preferred embodiment, the measuring device at least partly consists of an optically transparent material, so that optical measurements can be performed. For example, it can be examined whether the cell sends an optical signal upon the addition of a potential receptor ligand.

The invention relates to a process for establishing properties of cells and vesicles using the measuring device according to the invention.

Preferably, said at least one established property of the cells or vesicles is an optical or electrophysiological property of the cell membrane. For example, the electric resistance of the membrane, the current flow through the membrane and/or fluorescence signals can be measured according to the invention. In this way, not only properties of the cell membrane, but also the response of the cell membranes to external stimuli and changes, such as the addition of substances, can be examined. It is possible to establish individual values by point measurements, or to follow the change of parameters and thus to observe cellular processes. According to the invention, for example, it may be examined how ion channels in the cell membrane are opened or closed by the addition of a (potential) receptor ligand to the measuring device with the lipid bilayer and coupled cell.

In another embodiment, biological macromolecules can be contained in or associated with the lipid bilayer. Especially proteins, such as ion channels or receptors, are suitable.

In addition, after the cell has been fused with the lipid bilayer, examinations on both sides of the cell membrane are possible, depending on in which of the two chambers ligands are added, for example.

The vesicles have an outer membrane based on a lipid bilayer. The membrane may contain other usual components of biological membranes, such as proteins, but also artificially produced integrated components, such as recombinant proteins. In preferred embodiments, the vesicles are purified biological vesicles, such as cell organelles, for example, mitochondria, or artificially produced vesicles, such as liposomes or proteoliposomes. Vesicles according to the invention also include vesicular fractions of cells or organelle membranes.

The process according to the invention is suitable for automated performance, in particular. The invention also relates to an array for automatically establishing properties of cells or vesicles, consisting of at least two measuring devices according to claim 1. The measuring devices are preferably firmly connected to each other and can be individually addressed electrically. The arrays according to the invention allow the process to be performed at least partially by robots or machines. They comprise at least 2, preferably from 50 to 2000, especially 96, 384 or 1536 measuring devices (SBS standard).

The invention allows the utilization of microscopic horizontal lipid bilayers as supports for whole cells. These examination objects, which are important in the research into active substances, can be analyzed not only by the fluorescence-based methods already established in high-throughout screening (HTS), but also simultaneously by electrical analysis.

In addition, the individual support lipid bilayers can be miniaturized to a high degree and measured simultaneously or in close time relation in nanotechnologically structured and produced arrays (nanoarrays). The almost simultaneous examination of different pharmacologically relevant aspects, which is thus possible, can significantly shorten the overall process of screening for active substances. In addition, the novel technology will significantly extend the analysis of membrane transport processes. By this coupling of the cell and lipid bilayer, the electrical properties of almost any cell, vesicle or proteoliposome can be measured, for example.

In this way, the electrical and/or optical properties of the applied cell membranes can be measured and characterized in a reproducible and mechanically stable way. In contrast to the patch clamp technology, no patch pipettes, which require a stable sealing resistance, are used in this invention. Thus, applying a vacuum or reduced pressure in order to fix the cell or vesicle to the opening is not required either. Between the hydrophobic substrate and the lipid bilayer, the sealing resistance forms spontaneously and stably at least for hours. Further, this connection between the lipid bilayer and the hydrophobic sheet has the high mechanical stability which is necessary for HTS. The positioning of the cells or vesicles on the lipid bilayer is preferably effected simply by adding them above the lipid bilayer.

In addition, the method herein described is substantially less expensive than those known from and described by the prior art, since the measuring chambers can be produced in parallel in a sandwich style and do not contain any mechanically unstable components, such as vacuum lines.

Further, the optical path to the cell can be utilized without limitation, so that this invention can be combined with high-resolution fluorescence microscopy and spectroscopy.

The coupling between the lipid bilayer and cells or cellular vesicles, such as proteoliposomes, allows the electrical properties of the membranes to be measured at a sealing resistance of 10⁸-10¹⁰ Ω, for example. In addition, the process also allows parallel optical measurements. An exact positioning of the cell on the lipid bilayer is not necessary, so that the process is highly apt for automation and parallelization.

The invention also relates to single, double and triple measuring chambers as well as microtitration plates and processes according to any of claims 16 to 27. The single, double and triple measuring chambers according to the invention are particularly suitable for use in a process for the determination of cell properties according to the invention. However, they may also be used generally for examining properties of lipid bilayers without contacting cells.

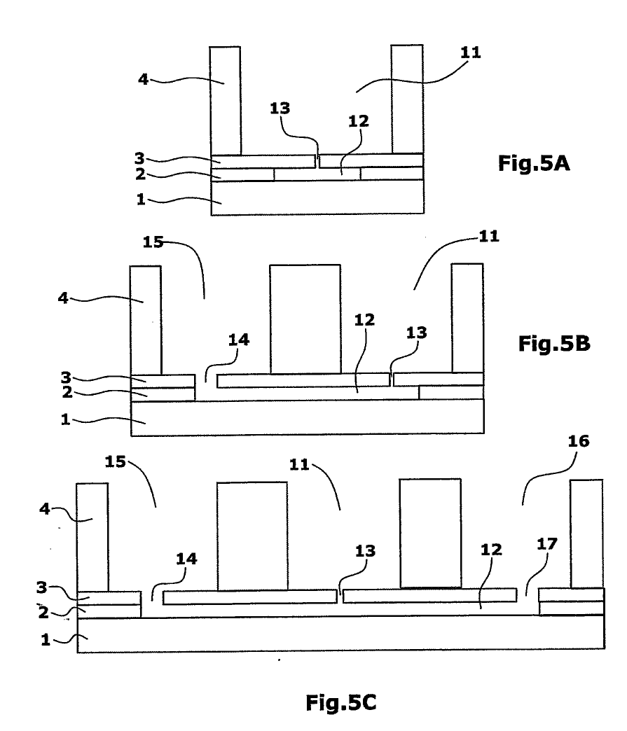

FIG. 5a shows a one-chamber system according to the invention comprising a first cavity (11) with lateral walls (4) and a partition wall (3), which is at the same time the bottom of the cavity. Below, a second cavity (12) is provided which is connected with said first cavity (11) through a measuring opening (13). Said second cavity (12) is laterally bounded by the layer (3) and by a lower bottom (1). Electrodes of metal are positioned in both cavities. The lower bottom can consist of an optically active material, such as glass.

FIG. 5b shows a double chamber system according to the invention comprising two first cavities (11) and (15) and a connecting second cavity (12), a first measuring opening (13) and a second opening (14).

FIG. 5c shows a triple chamber system according to the invention with the features according to FIG. 5b. In addition, another first cavity (16) with an opening (17) is contained. The second cavity (12) extends in a connecting manner below the first cavities (11), (15) and (16).

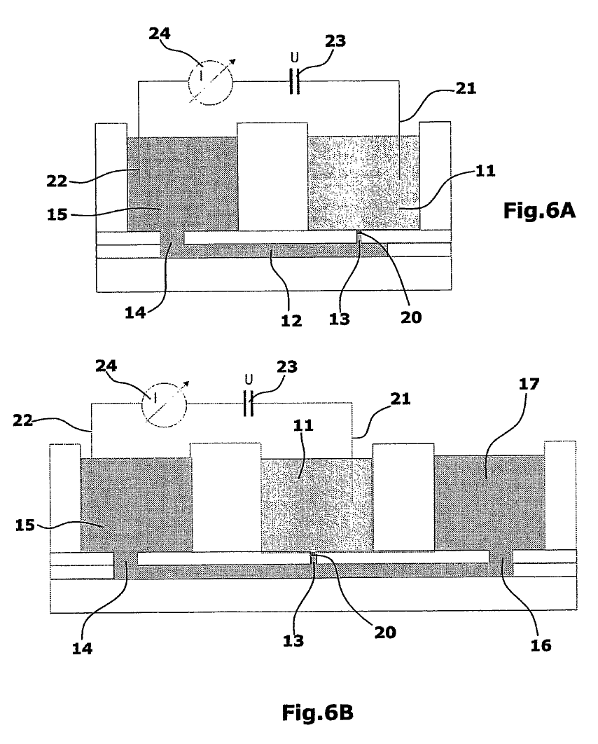

FIGS. 6a and b show the double and triple chamber system, respectively, according to the invention with the features according to FIGS. 5b and 5c. In addition, two electrodes (21) and (22) are contained which are connected via a voltage source U (23) and a current measuring device I (24). A lipid bilayer is positioned in the opening (13). The cavities are filled with liquid, wherein the lipid bilayer separates two solutions having different compositions.

FIG. 7 schematically shows how the exchange of liquid can be effected in particular compartments in the double or triple chamber systems with a lipid bilayer according to the invention. On the left side, the system before the exchange of liquid is respectively shown. The sites of withdrawal and addition are marked by bars with arrows. On the right-hand side, the system after partial exchange of the liquid is respectively shown, wherein the newly added liquid is shown in a lighter shade.

FIG. 8 shows how the double or triple chamber systems according to the invention can be arranged into arrays and microtitration plates. In FIG. 8a, a double measuring chamber unit connected with analogously arranged further double measuring chambers through a common bottom is indicated by boxing. Accordingly, a triple measuring chamber is indicated by boxing in FIG. 8b.

The single measuring chamber according to the invention, as illustrated in FIG. 5, comprises a first cavity (11) with lateral walls (4) and an upward opening. Below the first cavity (11), a second cavity (12) is positioned, wherein the first cavity is separated from the second cavity by a hydrophobic partition wall (3). The hydrophobic partition wall (3) has a measuring opening (13) with a diameter of from 0.1 to 100 μm. “Measuring opening” herein refers to an opening or hole into which a lipid bilayer can be introduced for subsequently measuring optical and electrical properties in the opening or its environment. In contrast, the openings (14, 17), which are not referred to as “measuring openings”, do not serve for receiving a lipid bilayer, but enable the liquid exchange and current flow between the first and second cavities. Therefore, they preferably have larger diameters than those of the measuring openings.

In the first and second cavities (11, 12), one electrode each made of wire is contained. The measuring chamber has such a design that a lipid bilayer which separated the first from the second cavity can be positioned in the measuring opening (13) by the above described processes. Through the two electrodes, a voltage can be applied across the lipid bilayer. To the first cavity (11), suitable liquids, such as electrolyte solutions and physiological buffer systems or cells to be examined can be added.

As illustrated in FIGS. 5B and 6A, a double measuring chamber according to the invention comprises two juxtaposed measuring chambers each of which has a first cavity (11, 15) with lateral walls (4) and upward openings. A connecting second cavity (12) is positioned below the two first cavities (11, 15). A first cavity (15) is connected with the second cavity (12) through an opening (14). The other first cavity (11) is separated from the second cavity (12) by a hydrophobic partition wall (3). Said partition wall (3) contains a measuring opening (13) having a diameter of from 0.1 to 100 μm, so that said first cavity (11) is connected with said second cavity (12). In each of the first cavities (11) and (15), an electrode (21, 22) is contained. In contrast to the single measuring chamber according to the invention, no electrode is provided in the second cavity (12). The second cavity (12) can be designed like a channel and simply filled and rinsed. The arrangement of the double measuring chamber enables a pressure compensation by changing the amounts of liquid in the cavities. Thus, it is avoided that the lipid bilayer is damaged or broken. The possibilities of adding and withdrawing liquid are schematically represented in FIG. 7A.

A triple measuring chamber according to the invention is illustrated in FIGS. 5C and 6B. It consists of a double measuring chamber with another first measuring chamber having a first cavity (16), a connecting second cavity (12) being present below the three first cavities (11, 15, 16), and the first cavity (16) being connected with the second cavity (12) through an opening (17). The lipid bilayer is positioned in the measuring opening (13) which separates the first cavity (11) from the second cavity (12).

This arrangement has the advantage that, after the lipid bilayer has formed and thus the cavity (11) has been separated, there is a communicating liquid system over the cavities 12, 15 and 16 through which the pressure is compensated. In addition, the liquid in the second cavity (12) can be exchanged (rinsed) in the presence of the lipid bilayers. This enables, for example, the continuous or stepwise change of the ion concentration, the rinsing and cleaning of the cavity (12), or the addition or withdrawal of additives whose effect on the lipid bilayer or on a contacting cell is examined. FIG. 7B schematically shows how the liquid in the middle first cavity can be exchanged in the presence of the lipid bilayer in a triple chamber system according to the invention. In the middle first cavity (cis), addition and withdrawal are effected by the same route, while in the two interconnected first cavities, the addition and withdrawal may be effected in different cavities, and the pressure compensation is effected through the lower cavity.

The pressure compensation also prevents the lipid bilayers from being changed or even damaged due to the osmotic gradient and thereby distort the measuring result. In the devices according to the invention, it is quite generally preferred that a pressure compensation is effected because the lower cavity (12) has at least one opening which is not closed by a bilayer.

According to the invention, the measuring chambers can be arranged to form microtitration plates. The microtitration plates of the present invention have at least two measuring chambers according to the invention. Preferably, the measuring chambers are identically arranged in rows in two directions of space (FIG. 8). In a preferred embodiment, the chambers contain an optical access from below, for example, by means of a window of silica glass, and a pipetting access from above. The preparation of the chambers can be effected in an inexpensive sandwich style. This ensures the disposable approach as required for pharmacological experiments. With the devices according to the invention, it is possible to perform both electrical and optical measurements in high-throughput on horizontal membranes simultaneously and individually while the SBS-conforming microtitration plates are retained. Parallelized measuring chambers in two directions of space can accommodate a large number in a small space.

The measuring chambers in the Figures are shown merely in schematic representation; in particular, the size relations of the components in the Figures do not necessarily correspond to the real relations. Preferably, in an arrangement according to FIG. 5, the bottom (1) has a vertical diameter of 5-200 μm, especially 10-100 μm, the layer (2) has a diameter of 5-200 μm, especially 10-100 μm, and the partition wall (3) has a diameter of 1-100 μm, especially 5 to 25 μm. The height of the lateral walls (4) is preferably from 0.5 to 10 mm, especially from 1 to 5 mm, and the horizontal diameter of the first cavity is from 0.5 to 10 mm, especially from 1 to 5 mm. The first cavities preferably have a capacity of 1 μl to 1 ml of liquid. The second cavity (12) preferably has the same vertical diameter as the layer (2). The width of the second cavity can vary, preferably between 0.1 and 500 μm.

Measurements of the electrophysiological properties of “membrane probes” and membrane proteins, especially membrane transporters, are increasingly gaining importance in pharmacological studies. In particular, it is of interest to compare the electrical properties of the membrane proteins with their optical ones. From the prior art, the method of Borisenko et al. (2003) is known for examining lipid bilayers. On the bottom side of a hydrophobic sheet, a thin conductive layer (e.g., silver) which leaves the optical access free and serves as an electrode in the case of electrical measurements is applied by vapor deposition. If only optical measurements are intended, the silver layer can be dispensed with. The measuring chamber has drawbacks in that the silver layer affects optical measurements and the existing agarose layer is prepared relatively complexly and, in addition, will degenerate by water loss within a period of several hours. Therefore, the device is not suitable, in particular, as a base for an automated method. Microtitration plates having volumes in the microliter scale (microtitration plates) are needed in the usual high-throughput screening (HTS) and ultrahigh-throughput screening (UHTS) experimental series in the search for active substances in pharma research. For known electrical and coupled electro-optical measuring methods, measuring chambers suitable for HTS and UHTS according to SBS (Society for Biomolecular Screening) standard are lacking.

The single, double and triple measuring chamber as well as microtitration plates of the invention are particularly suitable for establishing properties of cells or vesicles by the processes according to the invention and for preparing the measuring devices according to the invention in which at least one cell or one vesicle contacts the lipid bilayer. Therefore, the embodiments of the measuring devices according to the invention comprising vesicles or cells are in particular features of the single, double or triple measuring chambers of the invention.

The measuring chambers and microtitration plates according to the invention have numerous advantages over the known devices for examining lipid bilayers. The measuring chambers according to the invention allow to follow optically and/or electrically processes between two aqueous phases separated by a horizontal lipid bilayer in a process according to the invention. The use of miniaturized measuring chambers as well as the use of a sandwich construction in which a high number of measuring chambers with a low number of components are prepared is enabled. Optical access through the bottom of the device is ensured. Pipette and electrode access from above is ensured, so that the measuring method can be performed simply and also as an automated method.

By means of usual SBS-conforming microtitration plates, only changes within the lumen of a chamber can be essentially observed with optical methods. In contrast, the microtitration plate according to the invention offers the possibility to perform optical and electrical measurements simultaneously or individually in two aqueous compartments separated by a horizontal lipid bilayer membrane. Thus, the electrical and optical properties can be individually established in the various cavities. Further, it is possible to measure also optically and electrically transport processes through the membrane as well as processes related to the association to the membrane, dissociation or incorporation of effectors into the membrane. In contrast to conventional microtitration plates, the microtitration plates according to the invention offer the possibility to measure properties of a membrane or membrane processes electrically and/or optically without losing the character of a UHTS measuring chamber plate.

In the devices according to the invention, at least two cavities are separated by a lipid bilayer. Thus, the electrical parameters of transport processes can be measured across this membrane. In order to ensure optical access, this bilayer should be stabilized sufficiently closely above the glass plate, i.e., within focal depth. The structure is preferably a sandwich structure of different suitable materials, such as Teflon and glass. Such a layer structure is shown, for example, in FIG. 1 (layers 1, 2, 3 and 4).

The structure with juxtaposed measuring chambers to form a double or triple measuring chamber reduces the original number of measuring chambers by at least a factor of 2 (i.e., a 96-well microtitration plate becomes a 48-well double-well plate, etc.). However, this multiple chamber structure has the advantage that, after the chamber systems have been separated by a horizontal lipid bilayer, pressure compensation can be effected between the chamber systems without deforming the lipid bilayer (FIG. 7). This is essential to working with ion gradients between the two chambers.

A preferred embodiment for a double and triple measuring chamber according to the invention can be seen from FIG. 5. For example, one (or more) microstructured sheet (or sheets) has (have) been introduced between the upper portion of the measuring chambers and the glass bottom. By such sheet, at least two adjacent chambers are connected to form a double chamber (triple chamber etc., microtitration plate). Per double chamber, one hole having a small diameter (0.1-100 μm) as well as one hole with a preferably larger diameter and a cavity (channel) horizontally connecting the two holes are introduced into the sheet. For example, such cavity can be integrated in the sheet or in the silica glass. In a preferred embodiment, the separating membrane is arranged horizontally within the focal depth above the glass layer in a microtitration plate.

The single measuring chamber structure according to the invention with two cavities lying on top of one another does not reduce the number of chambers when arranged to a microtitration plate. For reasons of stability, it should be used mainly with symmetric or similar ion concentrations in both cavities because a pressure compensation between the cavities is possible only by deforming the membrane.

Of course, it is also possible to connect further measuring chambers with the triple measuring chambers according to the invention to produce systems having at least four measuring chambers interconnected by a common chamber/opening system; the invention relates to such systems as triple chamber systems with additional measuring chambers.

### EXAMPLE

In a plastic septum made of a Teflon sheet 25 μm in thickness which separates the two compartments, a hole of about 30-50 μm in diameter has been produced by an electrical process. In this hole, an artificial lipid bilayer is produced between the two aqueous compartments as previously described (Hinnah et al., 2002). Both compartments are contacted electrically by Ag⁺/AgCl electrodes. The measuring set-up of the chamber has such dimensions that the bilayer can be established in the focus of an epifocal or confocal fluorescence microscope (FIG. 1). Such a bilayer typically has a sealing resistance of >5 GΩ and upon application of a voltage gate (V_{cmd}) shows the current response as outlined in the scheme according to FIG. 1 in which the mean current at V_{cmd}=100 mV is <20 pA after the bilayer capacity has been charged.

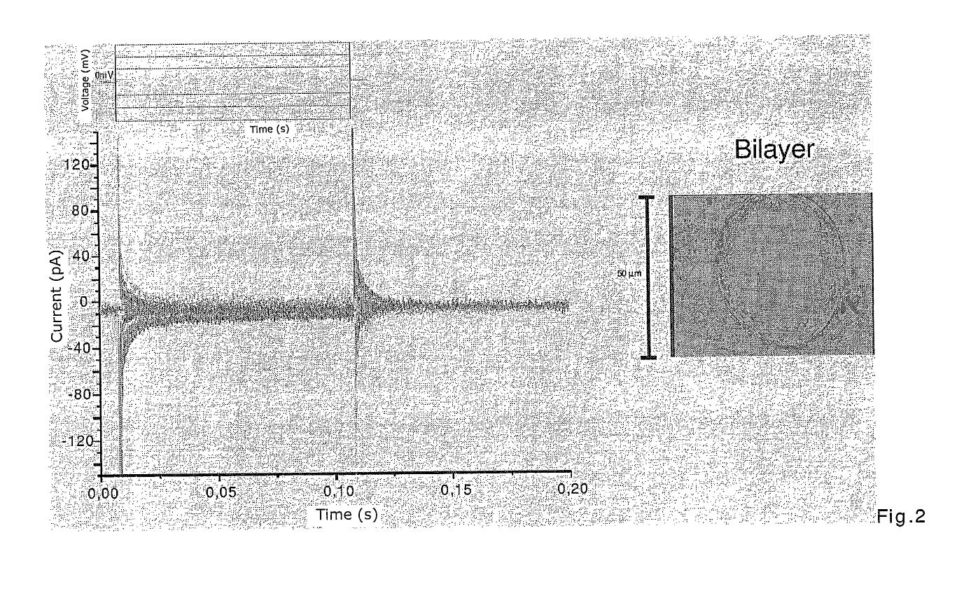

FIG. 2 shows voltage clamps on a horizontal bilayer (right panel) upon the application of voltage pulses (“voltage gate, top left panel). The amplitude of voltage (Vcmd) was increased in increments of V_{cmd}=±10 mV, starting from V_{cmd}=0 mV until V_{cmd}=100 mV (FIG. 2, top left). The bottom left Figure shows the time course of the current response weighted by the capacity and the resistance of the bilayer. It is characterized by two capacitive peaks (I·dt=C_{bilayer}·V_{cmd}) and the constant current (I=V_{cmd}/R_{bilayer}). FIG. 2 shows the real case in which the bilayer has a diameter of 50 μm and a mean sealing resistance of 9.5 GΩ.

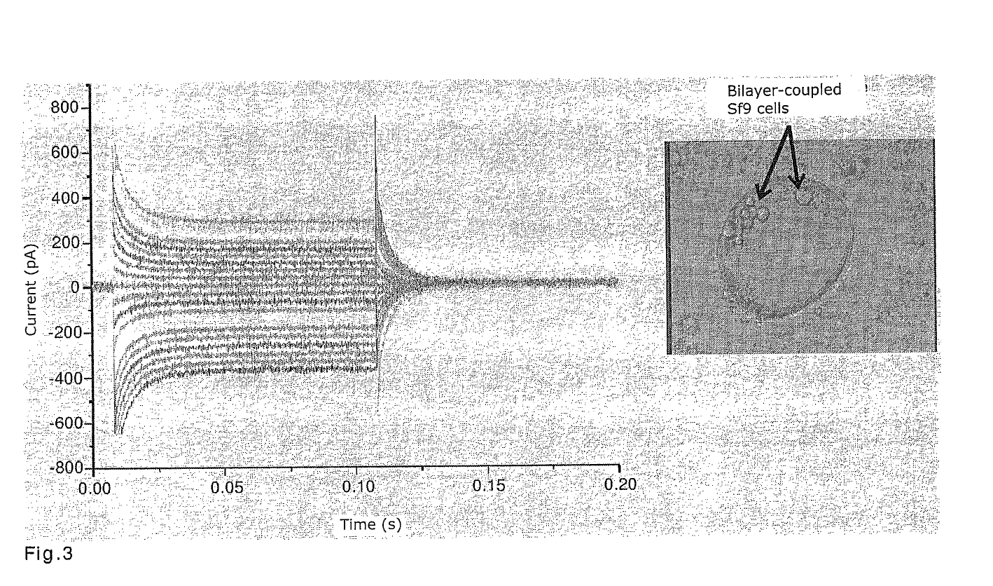

To this bilayer, at least one SF-9 cell is applied (FIG. 3). The depositing and manipulation of single or several cells is effected by micropipetting and micromanipulators. Then, upon capacitive or conductive coupling, the current response shown in FIG. 3 with significantly (>40 times as compared with FIG. 2) higher mean currents is measured when voltage gates are applied. SF-9 cells are a cell line derived from insects (Spodoptera frugiperda) used for the heterologous expression of proteins.

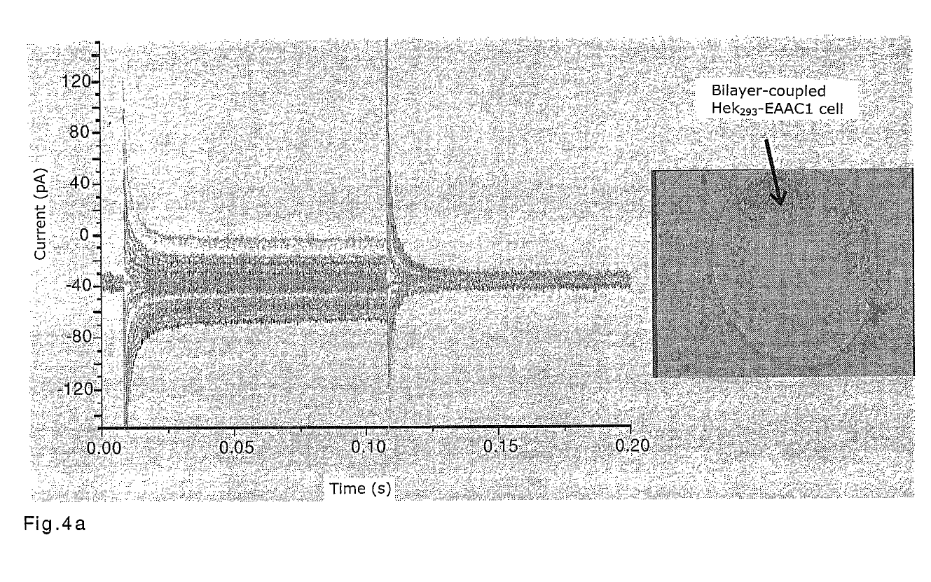

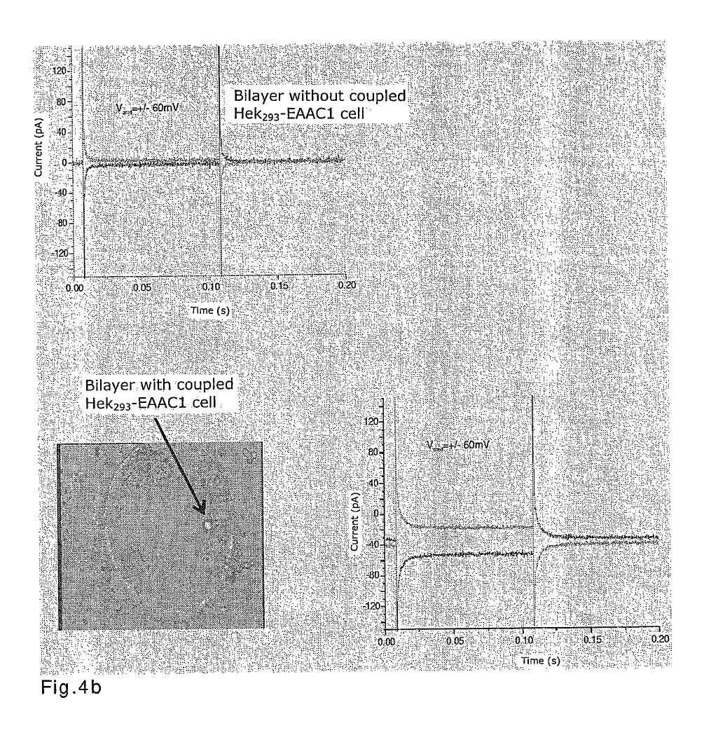

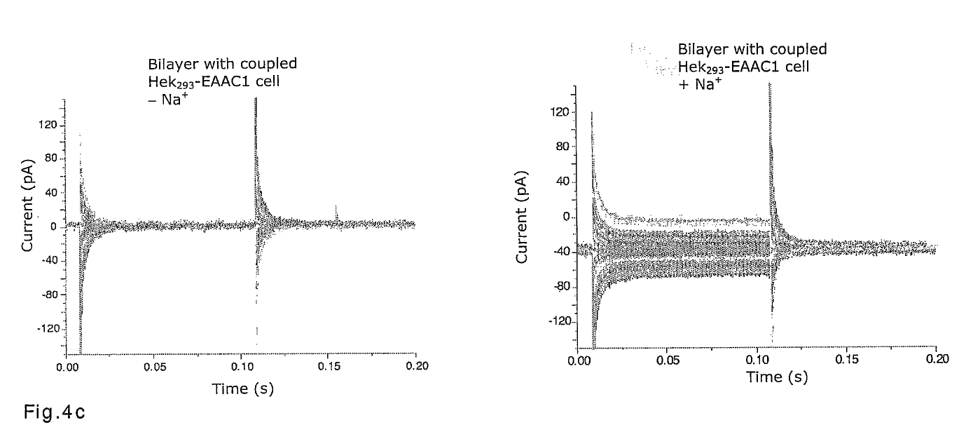

The quantitative verification of the coupling of cells on a horizontal bilayer by means of an Na⁺-dependent glutamate transporter (EAAC1) heterologously expressed in Hek cells is shown in FIG. 4. FIG. 4a shows a horizontal bilayer with an EAAC1 Hek₂₉₃ cell and the current response of this measuring set-up. A quantitative evaluation of the current response at V_{cmd}=±60 mV (FIG. 4b) and a comparison of the mean currents with the mean currents obtained in the same cell preparation in parallel experiments in a “whole-cell” patch clamp configuration (Grewer et al., 2000) are compared in Table 1.

TABLE 1

| | Patch clamp | Bilayer |
|---|---|---|
| | whole cell | coupled Hek₂₉₃-EAAC1 cell |
| | Iₘₑₐₙ in pA | Iₘₑₐₙ in pA |
| 0 mV | −45 | −35 |
| +60 mV | −10 | −15 |
| −60 mV | −115 | −56 |
| −Na⁺ | 0 | 0 |

The glutamate transporter is strictly Na⁺-dependent in its activity (Grewer et al., 2000). After the Na⁺ ions have been removed by perfusion in both aqueous compartments, the mean current is reduced to the value of bilayers without a coupled cell (FIG. 4c). Upon renewed addition of Na⁺ ions, the mean currents of the starting measuring set-up are reached (FIG. 4c).

These data show that the electrical current response obtained with the “whole cell” patch clamp set-up on Hek₂₉₃-EAAC1 cells is identical with that obtained from horizontal bilayers with a cell coupled thereon. The variations of the mean currents (Iₘₑₐₙ, Table 1) are to be attributed to the biochemically demonstrated variations of the expression rates of EAAC1 in Hek293 cells. These results demonstrate that the Hek₂₉₃-EAAC1 cells are coupled with the horizontal bilayer by capacitive and conductive coupling. The advantage of the process resides in the fact that in a horizontal set-up two aqueous compartments can be quickly and reproducibly separated by a lipid bilayer with a high resistance (>5 GΩ). The application of one or more cells to this bilayer as well as the application or fusion of macromolecules to or into this bilayer membrane allows to examine by optical and electrical measuring methods the transport and binding of charged and uncharged molecules through or to the cell membrane and correspondingly through the lipid bilayer membrane. Due to its properties, the process in highly suitable for parallel high-throughput screening measurements.

### References

Borisenko V, Lougheed T, Hesse J, Fureder-Kitzmuller E, Fertig N, Behrends J C, Woolley G A, Schutz G J. (2003) Simultaneous optical and electrical recording of single gramicidin channels. Biophys J 84(1): 612-22

Grewer C, Watzke N, Wiessner M, Rauen T. (2000) Glutamate translocation of the neuronal glutamate transporter EAAC1 occurs within milliseconds. Proc Natl Acad Sci U S A. 97(17):9706-11

Hinnah, S. C., Wagner, R., Sveshnikova, N., Harrer, R., and Soll, J. (2002) The chloroplast protein import channel Toc75: pore properties and interaction with transit peptides, Biophys. J. 83, 899-911.

Samsonov, A. V., Chatterjee, P. K., Razinkov, V. I., Eng, C. H., Kielian, M., Cohen, F. S. (2002) Effects of membrane potential and sphingolipid structures on fusion of Semliki Forest Virus, 3. Virol., 76, p 12691-12702.

Hanyu, Y., Yamada, T., Matsumoto, G. (1998) Simultaneous measurement of spectroscopic and physiological signals from a planar bilayer system: Detecting voltage-dependent movement of a membrane-incorporated peptide, Biochem., 37, p 15376-15382.

Bellamare, F., Dube, L., Sauve, R., (1996) Evidence from incorporation and patch-clamp experiments for a non-selective channel of large conductance at the luminal membrane of rabbit proximal tubule, Can. J. Physiol. Pharmacol., 74, p 265-272.

Sanford, M. S., Blobel, G., Zimmerberg, J. (1989) Large aqueous channels in membrane vesicles derived from the rough endoplasmic reticulum of canine pancreas or the plasma membrane of E. coli, Proc. Natl. Acad. Sci. USA, 86, 6176-6180.

1-27. (canceled)

28. A device for measuring at least one property of cells or vesicles, comprising:
(i) a hydrophobic partition wall forming two chambers in said device, and containing a horizontal opening into said two chambers;
(ii) an electrolyte solution contained in said two chambers;
(iii) a lipid bilayer that spans across said horizontal opening; and
(iv) at least one cell or vesicle in contact with said lipid bilayer.

29. The device of claim 28, wherein a membrane of said at least one cell or vesicle fuses at least partially with said lipid bilayer.

30. The device of claim 28, wherein said horizontal opening has a diameter of from 0.1 μm to 100 μm.

31. The device of claim 28, wherein said hydrophobic partition wall is made of a polymer film.

32. The device of claim 28, wherein said hydrophobic partition wall is made of Teflon, polycarbonate, polymethyl methacrylate (PMMA), polydimethylsiloxane (PDMS) or Topas.

33. The device of claim 28, wherein said lipid bilayer is made of synthetic or purified biological lipids.

34. The device of claim 28, wherein a circuit is applied through said device.

35. The device of claim 28, further comprising:
(v) an optically transparent material.

36. The device of claim 28, wherein said at least one property is an optical or electrophysiological property of a cell membrane.

37. The device of claim 28, wherein said vesicles are cell organelles, proteoliposomes, or vesicular fractions of cells or organelle membranes.

38. The device of claim 28, wherein biological macromolecules are contained in or associated with said lipid bilayer.

39. A method for establishing properties of cells or vesicles comprising:
(i) providing a device for measuring at least one property of cells or vesicles, comprising:
(a) a hydrophobic partition wall forming two chambers in said device, and having a horizontal opening into said two chambers;
(b) an electrolyte solution contained in said two chambers;
(c) a lipid bilayer that spans across said horizontal opening; and
(d) at least one cell or vesicle in contact with said lipid bilayer; and
(ii) measuring the optical or electrophysiological properties of said at least one cell or vesicle.

40. The method of claim 39, wherein said method is performed as an automated process.

41. An array for use in an automated process for establishing properties of cells or vesicles, comprising at least two devices of claim 28.

42. A device for measuring at least one property of cells or vesicles, comprising:
two juxtaposed measuring chambers, each of which comprising:
(i) a first cavity comprising lateral walls and an electrode;
(ii) a second cavity positioned below said first cavity; and
(iii) a hydrophobic partition wall separating said first cavity from said second cavity,
wherein said hydrophobic partition wall has an opening with a diameter of from 0.1 μm to 100 μm.

43. The device of claim 42, further comprising:
a third measuring chamber, comprising:
(i) a first cavity comprising lateral walls and an electrode;
(ii) a second cavity positioned below said first cavity; and
(iii) a hydrophobic partition wall separating said first cavity from said second cavity,
wherein said hydrophobic partition wall has an opening with a diameter of from 0.1 μm to 100 μm.

44. The device of claim of claim 42, wherein said opening has a diameter of from 0.5 μm to 5 μm.

45. The device of claim 42, wherein said hydrophobic partition wall is made of a polymer film.

46. The device of claim 42, wherein said hydrophobic partition wall is made of Teflon, polycarbonate, polymethyl methacrylate (PMMA), polydimethylsiloxane (PDMS) or Topas.

47. The device of claim 42, further comprising:
(iv) a lipid bilayer that spans across said opening.

48. The device of claim 47, wherein said lipid bilayer is made of synthetic or purified biological lipids.

49. The device of claim 42, wherein a circuit is applied through said device.

50. The device of claim 42, wherein the bottom of said second cavity is made of an optically transparent material.

51. A microtitration plate comprising at least two devices of claim 42, wherein said second cavities are interconnected.

52. A method for establishing properties of cells or vesicles, comprising:
(i) providing the device of claim 47; and
(ii) measuring the optical or electrophysiological properties of at least one cell or vesicle in contact with said lipid bilayer.

53. The method of claim 52, wherein said method is performed as an automated process.
